(19) United States
(12) Patent Application Publication
(10) Pub. No.: US 2013/0095060 A1
(43) Pub. Date: Apr. 18, 2013

(54) PHARMACEUTICAL COMPOSITION FOR PROMOTING ARTERIOGENESIS, AND PREPARATION METHOD AND APPLICATIONS FOR THE SAME

(71) Applicants: PATRICK C.H. HSIEH, TAINAN CITY (TW); YI-DONG LIN, TAICHUNG CITY (TW)

(72) Inventors: PATRICK C.H. HSIEH, TAINAN CITY (TW); YI-DONG LIN, TAICHUNG CITY (TW)

(73) Assignee: National Cheng Kung University, Tainan City (TW)

(21) Appl. No.: 13/443,073

(22) Filed: Apr. 10, 2012

(30) Foreign Application Priority Data
Oct. 12, 2011 (TW) 100136913

(51) Int. Cl. A61K 38/18; A61K 38/30; A61P 9/00; A61P 9/04; A61P 25/00; A61K 38/19; A61P 9/10

(52) U.S. Cl. 424/85.1; 514/8.1; 514/9.1; 514/9.5; 514/9.6; 514/8.4; 514/8.9; 514/8.2; 514/8.5; 514/9.2; 514/8.8; 514/7.7; 514/7.6

(57) ABSTRACT

The present invention relates to a pharmaceutical composition for promoting arteriogenesis, and preparation method and applications of the same, wherein said pharmaceutical composition comprises an effective amount of a drug, and a peptide hydrogel, and it forms a microenvironment for autologous cell recruitment and tissue regeneration.

### BACKGROUND OF THE INVENTION

1. Field of the Invention

The present invention relates to a pharmaceutical composition for promoting arteriogenesis, and preparation method and applications of the same.

2. Description of the Related Art

Cardiovascular disease is the leading cause of death in advanced countries of the world including in the US.¹ In Taiwan, about two million patients suffer from cardiovascular diseases and 0.4 million patients die from heart failure every year among the 23 millions of population, and most of these patients have irreversible damages caused by coronary artery disease and shortage of blood supply, such as myocardial loss, pathological remodeling, cardiac dysfunction and heart failure.^{2, 3}

Angiogenic therapy is a promising approach for tissue repair and regeneration. However, recent clinical trials using protein delivery or gene therapy to promote angiogenesis have failed to provide therapeutic effects. The key factors for achieving effective revascularization are the durability of the microvasculature and the process of arteriogenesis. Vascular endothelial growth factor (VEGF) plays an important role in angiogenesis and vasculogenesis.⁴⁻⁶ Thus, the application of VEGF in treatment of ischemic diseases has attracted great attention. In many recent studies, VEGF is delivered into vessels or the myocardium to promote angiogenesis. However, VEGF markedly increases vascular permeability of many organs in subjects, causes protein leakage, and induces general edema and immediate hypotension, tissue damage, even cancer or cancer metastasis. The development of using VEGF for cardiovascular regeneration is limited because there still lacks a proven technology which provides a controlled local delivery for clinical use in myocardial lesions, and most clinical studies using VEGF for treating ischemic diseases do not have promising results. Recent studies have demonstrated that the VEGF therapy is challenged by (1) the ability to maintain the local VEGF concentration at an effective level⁷⁻¹¹ and (2) an unavoidable vessel regression within 2 weeks after VEGF delivery due to the lack of additional regulation.^{7, 12-14} To overcome these pitfalls, it is crucial to provide an extracellular matrix for recruiting mural cells, such as pericytes or smooth muscle cells, to envelope and stabilize the nascent fragile endothelial tubes and subsequently achieve capillary stability and durable arteriogenesis.¹²⁻¹⁵

Recent developments in nanotechnology and biomaterials have generated the potential to recapitulate the biomimetic milieu in vitro¹⁶⁻¹⁹. In regenerative medicine, it is important to successfully generate in vivo tissue microenvironments with curative efficacy²⁰⁻²², and to stimulate the repair process by a natural manner. Therefore, the creation of intramyocardial microenvironments with sufficient VEGF concentrations and arteriogenic support will provide a novel method for effectively treating ischemic diseases.

### SUMMARY OF THE INVENTION

Accordingly, the inventors have developed a novel approach through an intramyocardial injection of peptide nanofiber hydrogel combined with VEGF, which significantly improves post-infarction angiogenesis, arteriogenesis and cardiac performance, in both rat and pig models of myocardial infarction (MI). This approach not only allows a local, controlled delivery in the myocardium to prolong the effect of VEGF without systemic harmful effects, but also creates a microenvironment favorable for recruiting myofibroblasts and bone marrow cells, allowing them to infiltrate and mature. Strikingly, this microenvironment further attracts a population of cardiomyocyte-like cells to the injection sites, suggesting cardiac regeneration is induced.

The objective of the present invention is to provide a pharmaceutical composition for promoting arteriogenesis, and preparation method and applications of the same. The pharmaceutical composition is obtained by combining VEGF and self-assembling peptide nanofibers (NFs). When the pharmaceutical composition is injected into the ischemic lesion of myocardial infarction, a slow, gradual, local low-dose drug delivery is achieved. After that, the capillary density of the myocardium is increased, and the arteriogenesis and the potential myocardium generation are promoted without side effects such as general edema and proteinuria. Therefore, the pharmaceutical composition of the present invention has an extreme potential for clinical use.

To achieve the goal, the present invention provides a self-assembling peptide, which is a repeating sequence with alternating hydrophobic and hydrophilic amino acids, and having 8-200 amino acids in length; more preferably, 12-32 amino acids; and even more preferably, 16 amino acids. The peptides of the present invention are complementary (i.e., ionic or hydrogen bonds are formed between peptides) and structurally compatible (i.e. a distance is maintained between peptide chains). The examples of the self-assembling peptide of the present invention is given as below:

(SEQ ID NO: 1)
AcN-AKAKAEAEAKAKAEAE-NH2;
(SEQ ID NO: 2)
AcN-AKAEAKAEAKAEAKAE-NH2;
(SEQ ID NO: 3)
AcN-EAKAEAKAEAKAEAKA-NH2;
(SEQ ID NO: 4)
AcN-KAEAKAEAKAEAKAEA-NH2;
(SEQ ID NO: 5)
AcN-AEAKAEAKAEAKAEAK-NH2;
(SEQ ID NO: 6)
AcN-ADADARARADADARAR-NH2;
(SEQ ID NO: 7)
AcN-ARADARADARADARAD-NH2;
(SEQ ID NO: 8)
AcN-DARADARADARADARA-NH2;
(SEQ ID NO: 9)
AcN-RADARADARADARADA-NH2;
(SEQ ID NO: 10)
AcN-ADARADARADARADAR-NH2;
(SEQ ID NO: 11)
AcN-ARADAKAEARADAKAE-NH2;
(SEQ ID NO: 12)
AcN-AKAEARADAKAEARAD-NH2;
(SEQ ID NO: 13)
AcN-ARAKADAEARAKADAE-NH2;
(SEQ ID NO: 14)
AcN-AKARAEADAKARADAE-NH2;
(SEQ ID NO: 15)
AcN-AQAQAQAQAQAQAQAQ-NH2;
(SEQ ID NO: 16)
AcN-VQVQVQVQVQVQVQVQ-NH2;
(SEQ ID NO: 17)
AcN-YQYQYQYQYQYQYQYQ-NH2;
(SEQ ID NO: 18)
AcN-HQHQHQHQHQHQHQHQ-NH2;
(SEQ ID NO: 19)
AcN-ANANANANANANANAN-NH2;
(SEQ ID NO: 20)
AcN-VNVNVNVNVNVNVNVN-NH2;
(SEQ ID NO: 21)
AcN-YNYNYNYNYNYNYNYN-NH2;
(SEQ ID NO: 22)
AcN-HNHNHNHNHNHNHNHN-NH2;
(SEQ ID NO: 23)
AcN-ANAQANAQANAQANAQ-NH2;
(SEQ ID NO: 24)
AcN-AQANAQANAQANAQAN-NH2;
(SEQ ID NO: 25)
AcN-VNVQVNVQVNVQVNVQ-NH2;
(SEQ ID NO: 26)
AcN-VQVNVQVNVQVNVQVN-NH2;
(SEQ ID NO: 27)
AcN-YNYQYNYQYNYQYNYQ-NH2;
(SEQ ID NO: 28)
AcN-YQYNYQYNYQYNYQYN-NH2;
(SEQ ID NO: 29)
AcN-HNHQHNHQHNHQHNHQ-NH2;
(SEQ ID NO: 30)
AcN-HQHNHQHNHQHNHQHN-NH2;
(SEQ ID NO: 31)
AcN-AKAQADAKAQADAKAQAD-NH2;
(SEQ ID NO: 32)
AcN-VKVQVDVKVQVDVKVQVD-NH2;
(SEQ ID NO: 33)
AcN-YKYQYDYKYQYDYKYQYD-NH2;
(SEQ ID NO: 34)
AcN-HKHQHDHKHQHDHKHQHD-NH2;
(SEQ ID NO: 35)
AcN-RARADADARARADADA-NH2;
(SEQ ID NO: 36)
AcN-RADARGDARADARGDA-NH2;
(SEQ ID NO: 37)
AcN-RAEARAEARAEARAEA-NH2;
(SEQ ID NO: 38)
AcN-KADAKADAKADAKADA-NH2;
(SEQ ID NO: 39)
AcN-AEAEAHAHAEAEAHAH-NH2;
(SEQ ID NO: 40)
AcN-FEFEFKFKFEFEFKFK-NH2;
(SEQ ID NO: 41)
AcN-LELELKLKLELELKLK-NH2;
(SEQ ID NO: 42)
AcN-AEAEAKAKAEAEAKAK-NH2;
(SEQ ID NO: 43)
AcN-AEAEAEAEAKAK-NH2;
(SEQ ID NO: 44)
AcN-KAKAKAKAEAEAEAEA-NH2;
(SEQ ID NO: 45)
AcN-AEAEAEAEAKAKAKAK-NH2;
(SEQ ID NO: 46)
AcN-RARARARADADADADA-NH2;
(SEQ ID NO: 47)
AcN-ADADADADARARARAR-NH2;
(SEQ ID NO: 48)
AcN-DADADADARARARARA-NH2;
(SEQ ID NO: 49)
AcN-HEHEHKHKHEHEHKHK-NH2;
(SEQ ID NO: 50)
AcN-VEVEVEVEVEVEVEVEVEVE-NH2;
and
(SEQ ID NO: 51)
AcN-RFRFRFRFRFRFRFRFRFRF-NH2;

in which each of the above self-assembling sequences comprises a repeating motif sequence with 2-16 amino acids, and the same motif sequence can be repeated to obtain a sequence having up to 200 amino acids. The right end of all self-assembling sequences is not —CNH₂.

The present invention also provides a pharmaceutical composition for promoting arteriogenesis, comprising: (1) an effective amount of a drug for promoting arteriogenesis selected from the group consisting of vascular endothelial growth factor (VEGF), fibroblast growth factor (FGF), granulocyte colony-stimulating factor (GCSF), granulocyte-macrophage colony stimulating factor (GM-CSF), hepatocyte growth factor (HGF), angiopoietin, epidermal growth factor (EGF), nerve growth factor (NGF), transforming growth factor (TGF), platelet-derivated growth factor (PDGF), insulin-like growth factor (IGF), brain-derived neurotrophic factor (BDNF), keratinocyte growth factor (KGF), bone morphogenetic protein (BMP), erythropoietin (EPO) and placental growth factor (PIGF); and (2) a peptide hydrogel comprising the self-assembling peptide defined above; in which the pharmaceutical composition forms a microenvironment for autologous cell recruitment and tissue regeneration.

In a preferred embodiment, said self-assembling peptide of the pharmaceutical composition is AcN-RARADADARARADADA-NH₂ (SEQ ID NO: 35), represented by the following structural formula:

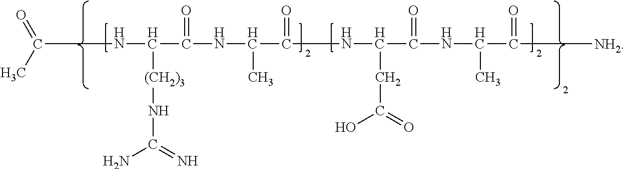

In a preferred embodiment, said peptide hydrogel is used as a carrier for loading said drug.

In a preferred embodiment, said peptide hydrogel is composed of said self-assembling peptide and a buffer solution. More preferably, said peptide hydrogel comprises 0.1% to 10% by weight of said self-assembling peptide; even more preferably, 0.5% to 5% by weight of said self-assembling peptide; most preferably, about 1% by weight of said self-assembling peptide.

In a preferred embodiment, said buffer solution is water, saline, or a buffer solution comprising elements needed for peptide self-assembly; more preferably, phosphate buffer solution.

In a preferred embodiment, said drug is physically connected with said peptide hydrogel.

In a preferred embodiment, said drug is vascular endothelial growth factor (VEGF); more preferably, VEGF₁₂₁, VEGF₁₄₅, VEGF₁₆₅; most preferably, VEGF₁₆₅.

In a preferred embodiment, said tissue regeneration is tissue regeneration of heart, liver, spleen, lung, kidney, brain, pancreas, eye, cartilage, urinary bladder or muscle.

In a preferred embodiment, said pharmaceutical composition is used to treat a cardiovascular disease; more preferably, said cardiovascular disease comprises myocardial infarction (MI), heart failure, ischemic heart diseases, stroke and peripheral vascular diseases; even more preferably, said cardiovascular disease is ischemic heart disease.

In a preferred embodiment, said pharmaceutical composition is administered by injection into the myocardium through thoracotomy, cardiac catheterization, echo-guided injection, or any other methods for injection.

In a preferred embodiment, said autologous cell is myofibroblast, bone marrow cell, cardiomyocyte-like cell, precursor cell and/or stem cell; more preferably, said precursor cell is vascular precursor cell or myocardial precursor cell, and said stein cell is vascular stem cell or myocardial stein cell.

The present invention also provides a method for preparing the above-mentioned pharmaceutical composition, comprising the following steps:
(1) preparing a peptide hydrogel from a self-assembling peptide and a buffer solution, wherein said self-assembling peptide is the self-assembling peptide defined above; and
(2) mixing an effective amount of a drug for promoting arteriogenesis and said peptide hydrogel to obtain the above-mentioned pharmaceutical composition, and said drug for promoting arteriogenesis is the drug for promoting arteriogenesis defined above.

In a preferred embodiment, said self-assembling peptide of the preparation method is AcN-RARADADARARADADA-NH₂ (SEQ ID NO: 35), represented by the following structural formula:

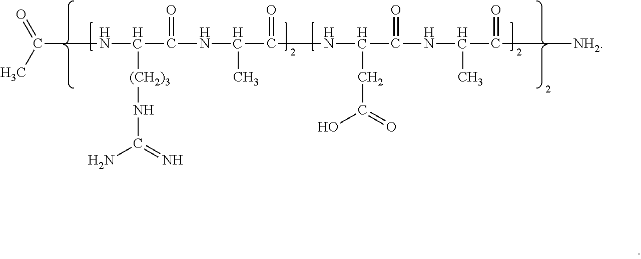

In a preferred embodiment, said peptide hydrogel of the preparation method comprises 0.1% to 10% by weight of said self-assembling peptide; more preferably, 0.5% to 5% by weight of said self-assembling peptide; most preferably, about 1% by weight of said self-assembling peptide.

In a preferred embodiment, said buffer solution of the preparation method is water, saline, or a buffer solution comprising elements needed for peptide self-assembly; more preferably, phosphate buffer solution.

In a preferred embodiment, said peptide hydrogel of the preparation method is prepared by mixing through sonication or any other process for mixing the peptide hydrogel in the step (1).

In a preferred embodiment, said drug of the preparation method is vascular endothelial growth factor (VEGF); more preferably, VEGF₁₂₁, VEGF₁₄₅, VEGF₁₆₅; most preferably, VEGF₁₆₅.

Yet, the present invention provides a method for treating a cardiovascular disease, comprising applying the above-mentioned pharmaceutical composition to a subject in need; more preferably, said cardiovascular disease comprises myocardial infarction, heart failure, ischemic heart diseases, stroke and peripheral vascular diseases.

From above, it should be clear that the pharmaceutical composition of the present invention provides a controlled local delivery of VEGF in the myocardium without increased systematic vascular permeability, in which side effects are dramatically reduced. In addition, it promotes angiogenesis of the myocardium, but also improves heart functions after infarction and reduces the infarction area. The preparation method of the present invention provides a sustained release, pharmaceutical acceptable carrier by simple steps, which can be used to control the local delivery of VEGF in the myocardium.

### BRIEF DESCRIPTION OF THE DRAWINGS

FIG. 1 represents the schematic illustration of the NF/VEGF composition of the present invention and their injection sites.

FIG. 2 represents the diagram showing that the NF/VEGF composition of the present invention promotes post-infarction replenishment of myofibroblast-derived mural cells and exogenous DiI-labeled bone marrow cells. (A) A histogram representing myofibroblasts (α-SMA⁺) at the border zone from each group on day 3, 7 or 14 post-MI. (B) A histogram representing bone marrow cells (DiI⁺) at the border zone of each group.

FIG. 3 shows that the NF/VEGF composition of the present invention increases the cardiomyocyte-like cell population (GFP⁻/cTnI⁺) on day 28 post-MI.

### DETAILED DESCRIPTION OF THE PREFERRED EMBODIMENTS

The inventors reveal for the first time that the self-assembling peptide nanofibers (NFs) can function as a vehicle for controlled local release of VEGF in the heart. Also, they prove that the NF/VEGF composition of the present invention can establish a microenvironment potent for vascular generation and endogenous myofibroblast recruitment to improve angiogenesis and arteriogenesis of myocardium after infarction. Therefore, the NF/VEGF composition of the present invention can improve cardiac performance after infarction and reduce the infarct area, and it has great potential for clinical use.

The examples of the present invention are provided hereinafter, however, these examples are not used for limiting the scope of the present invention.

### EXAMPLES

### Example 1

### Preparation of the NF/VEGF Composition of the Present Invention

SEQ ID NO: 35 (synthesized by SynBioSci, Livermore, Calif.) is used in the following Examples.

The powder of self-assembling peptide SEQ ID NO: 35 was formulated as a peptide solution of 1% by weight by phosphate buffered saline (PBS), pH 7.4, and sonicated (100 W, 10 minutes) into a peptide hydrogel of 1% by weight.

During the synthesis process of vascular endothelial growth factor (VEGF), a variety of VEGF protein forms are produced due to different mRNA splicing ways, such as VEGF₁₂₁, VEGF₁₄₅, VEGF₁₆₅, VEGF₁₈₅ and VEGF₂₀₆, in which VEGF₁₂₁, VEGF₁₄₅ and VEGF₁₆₅ are secreted, soluble protein forms that directly act on vascular endothelial cells to promote the proliferation of vascular endothelial cells and increase vascular permeability. The VEGF solution used in the following examples is VEGF₁₆₅ solution having a concentration of 1000 ng/mL in PBS (pH 7.4), but other VEGF protein forms also can be used.

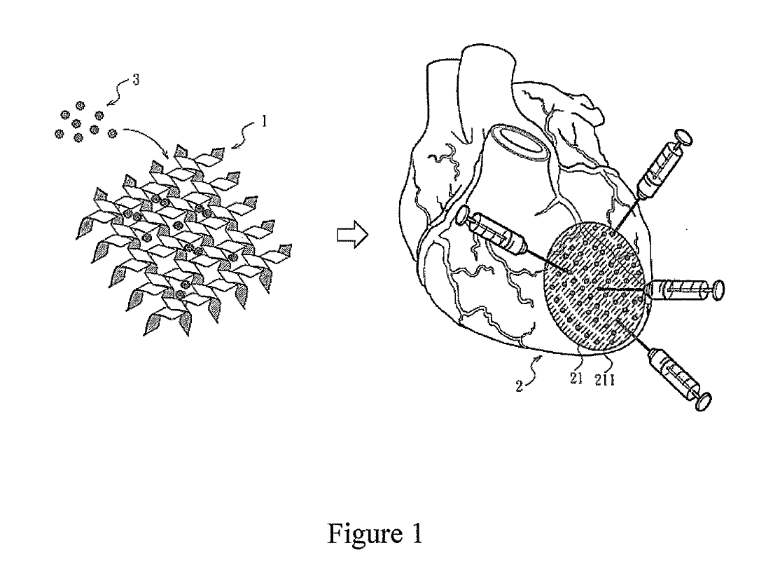

The above-mentioned VEGF solution and peptide hydrogel were mixed at a volume ratio of 1:9 to obtain the NF/VEGF composition of the present invention. As shown in FIG. 1, the composition comprises a self-assembling peptide (1) and VEGF (3).

### Example 2

### Vascular Permeability Study of VEGF

6-week-old male SD rats (250 g body weight) were randomly divided into 4 groups (n≧4 in each group) for the vascular permeability study of VEGF.

80 μL of (a) PBS, pH 7.4 (PBS group), (b) VEGF solution of Example 1 (VEGF group), (c) 1% by weight peptide hydrogel of Example 1 (NFs group) or (d) the NFs/VEGF mixture of Example 1 (NFs/VEGF group) was given by intramyocardial injection at 6 different injection sites (211) in the region which could be damaged by infraction (21) in a mouse heart (2). The FIG. 1 is a schematic illustration of the injection, in which the injection sites (211) are illustrative, not real injection sites. 45 minutes after the injection, 150 μL of red fluorescent FluoSpheres (Molecular Probes, Invitrogen) was injected into the left internal jugular vein to assess the vascular permeability. After 30 minutes of FluoSphere circulation, urine samples of the rats were collected. After that, systemic perfusion was performed to wash out the FluoSphere in the blood of rats. Following the perfusion, the organs such as brain, heart, lung, liver, spleen and kidney were collected for frozen embedding and further investigation. NanoDrop 2000 (Thermo) was used to analyze vascular permeability. Frozen tissue sections were stained, and the fluorescence strength was quantified by fluorescence microscope and image analysis software. Higher fluorescence strength indicates increased vascular permeability of the organ. The baseline (1x) was set by the data from the control group (PBS group), and the relative fluorescence strength in folds of the other groups against the control group were calculated, as shown in Table 1. In brain, for example, the fluorescence strength of VEGF group is 239 folds higher than the control group.

The urine samples were analyzed based on the protein concentration of the samples. Similarly, the baseline (1x) was set by the data from the control group (PBS group), and the relative protein concentration in folds of the other groups against the control group were calculated.

TABLE 1
Vascular permeability of rats following different treatment (in folds):

| Groups (n ≧ 4) | PBS | VEGF | NFs | NFs/VEGF |
|---|---|---|---|---|
| Brain | 1.0 ± 1.0 | 239.4 ± 93.4** | 0.8 ± 0.4 | 4.9 ± 1.5 |
| Heart | 1.0 ± 0.3 | 1.4 ± 0.3 | 1.7 ± 0.5 | 7.8 ± 2.5* |
| Lung | 1.0 ± 0.5 | 6.7 ± 2.7* | 0.2 ± 0.1 | 1.3 ± 0.9 |
| Liver | 1.0 ± 0.4 | 16.6 ± 5.6** | 2.1 ± 0.3 | 1.8 ± 0.6 |
| Spleen | 1.0 ± 0.4 | 16.1 ± 6.6* | 2.4 ± 0.5 | 1.8 ± 0.6 |
| Kidney | 1.0 ± 0.4 | 23.5 ± 5.9*** | 0.3 ± 0.2 | 2.2 ± 1.2 |
| Urine | 1.0 ± 0.1 | 1.8 ± 0.2* | 0.7 ± 0.2 | 0.9 ± 0.3 |

*P < 0.05,
**P < 0.01,
***P < 0.001, vs. PBS group.

From Table 1, it should be clear that when VEGF alone was given by intra-myocardial injection (VEGF group), the other organs (such as the brain, lung, liver, spleen and kidney) in the rats had a greater vascular permeability than the control group (PBS group), which reached 6˜240 folds of the control group. And, the protein concentration of urine samples of VEGF group was nearly twice higher than the control group. However, injection of the NF/VEGF composition of the present invention (NFs/VEGF group) significantly decreased the vascular permeability of these organs. It approximately reduced the vascular permeability caused by VEGF to 1˜5 folds of the control group.

On the contrary, injection of the NF/VEGF composition of the present invention (NFs/VEGF group) increased the vascular permeability of the hearts of rats to 7 folds of the control group. This result indicates that VEGF comprised in the NF/VEGF composition acts more effectively for the heart than for the other organs. In other words, the NF/VEGF composition of the present invention has the ability to achieve a controlled local delivery of VEGF in the target organ, the heart. This shows the NF/VEGF composition of the present invention has high safety and effectiveness.

### Example 3

### Sustained Release of VEGF in the Myocardium

6-week-old male SD rats (250 g body weight) were randomly divided into 7 groups for the following study for a sustained release of VEGF in the heart. Group 1 was sham operation group, in which the chest cavity of rats was opened without coronary artery ligation (n=8). In groups 2-4, the chest cavity of rats was opened with permanent ligation of the left anterior descending (LAD) coronary artery (experimental MI, for mimicking myocardial infarction), and the rats were injected with phosphate buffer solution (PBS), or 100 ng/mL or 1000 ng/mL VEGF solution in PBS (MI+PBS, MI+V100, MI+V1000 groups; n=8). In the groups 5-7, the chest cavity of rats was opened with permanent ligation of the LAD coronary artery (to mimic myocardial infarction, MI), and the rats were injected with 1% by weight of peptide hydrogel, or 100 ng/mL or 1000 ng/mL NF/VEGF composition of the present invention (MI+NFs, MI+NFs/V100, MI+NFs/V1000 groups; n=8). In all groups, the formulations including solution, hydrogel and composition were given by intramyocardial injection at 6 different sites in the infarct, and the volume of the formulations was 80 μL.

After the operation for mimicking myocardial infarction and formulation injection of groups 1˜7, left ventricular contraction function of rats was assessed by echocardiography on day 1 and 28 after the operation (i.e., post-MI) under M-mode, and the left ventricular ejection fraction (EF %) was assessed, as shown in Table 2.

The rats were sacrificed on day 28 post-MI, the hearts were collected, embedded in paraffin wax and sectioned, and the circumference of left ventricle wall and the length of infarcted left ventricular wall were measured. The size of infarcted area was calculated by the following formula, as shown in Table 2. The greater infarcted area indicates a greater damage of myocardium.

$$Ratio of infarct length = \frac{the length of infarcted left ventricular wall}{the circumference of left ventricle wall} \times 100 \%$$

TABLE 2
Cardiac function of rats on day 28 post-MI

| Groups (n ≧ 8) | EF (%) | Ratio of infarct length (%) |
|---|---|---|
| sham operation | 61.3 ± 2.0 | 0 |
| MI + PBS | 40.6 ± 1.9 | 64.4 ± 5.2 |
| MI + V100 | 43.9 ± 2.0 | 57.7 ± 4.3 |
| MI + V1000 | 45.9 ± 2.1 | 53.1 ± 9.0 |
| MI + NFs | 42.4 ± 2.5 | 48.5 ± 5.1 |
| MI + NFs/V100 | 50.4 ± 2.0** | 38.1 ± 2.8* |
| MI + NFs/V1000 | 49.9 ± 1.1** | 38.3 ± 7.6* |

*P < 0.05,
**P < 0.01, vs. MI + PBS group.

Although there was a marginal dose-dependent amelioration of the cardiac contraction functions in MI+V100 and MI+V1000 groups on day 28 post-MI against the control group, there was no significant difference in the size of infarct area between these VEGF-treated groups and the control group (MI+PBS). And, as the data shown in Table 2, the delivery of VEGF alone was not sufficient to improve the cardiac functions or to decrease the infarct size.

Similarly, when the peptide hydrogel of the present invention alone was injected (MI+NFs group), the cardiac contraction functions on day 28 post-MI were not improved. However, when the NF/VEGF composition of the present invention was injected (MI+NFs/V100 and MI+NFs/V1000 groups), the cardiac functions were improved and the infarct size was reduced.

Furthermore, immuno-staining was performed using antibody against isolectin (Invitrogen) to specifically label the endothelial cells in the sections, and antibody against SM22α (Abeam) to specifically label the smooth muscle cells in the sections. After that, the capillary and artery densities around the infarcted area of the myocardial sections were calculated, in which the artery was defined as arteriole if the diameter of vessel was less than 75 μm, and as artery if the diameter was greater than 75 μm. The results are shown in Table 3.

TABLE 3
Arteriogenesis indexes of rats on day 28 post-MI

| | Capillary density | Arteriole density | Artery density |
|---|---|---|---|
| Groups (n ≧ 8) | (capillaries/mm²) | (arterioles/mm²) | (arteries/mm²) |
| sham operation | 1208 ± 44.2 | 16.7 ± 3.6 | 2.4 ± 0.4 |
| MI + PBS | 127.4 ± 14.9 | 9.0 ± 2.4 | 1.5 ± 0.6 |
| MI + V100 | 260.0 ± 14.5** | 17.0 ± 3.8 | 3.4 ± 0.9 |
| MI + V1000 | 341.2 ± 15.9*** | 22.6 ± 4.9 | 4.2 ± 0.7 |
| MI + NFs | 218.7 ± 27.6* | 11.4 ± 2.2 | 2.1 ± 0.3 |
| MI + NFs/V100 | 355.1 ± 60.3*** | 44.5 ± 5.5*** | 6.6 ± 1.0*** |
| MI + NFs/V1000 | 368.5 ± 30.2*** | 39.3 ± 3.6*** | 7.3 ± 0.7*** |

*P < 0.05,
**P < 0.01,
***P < 0.001, vs. MI + PBS group.

In comparison with the control group (MI+PBS), the capillary densities of groups of VEGF, NFs and NFs/VEGF of the present invention were obviously improved on day 28 post-MI; however, the arteriole densities were improved only when the NF/VEGF compositions of the present invention were injected (MI+NFs/V100 and MI+NFs/V1000 groups), and the artery densities of these two groups were also higher than the other groups.

### Example 4

### NF Injection of the Present Invention Results in Myofibroblast Engraftment

Next, a study was conducted to explore the mechanism of arteriogenesis following NF/VEGF injection.

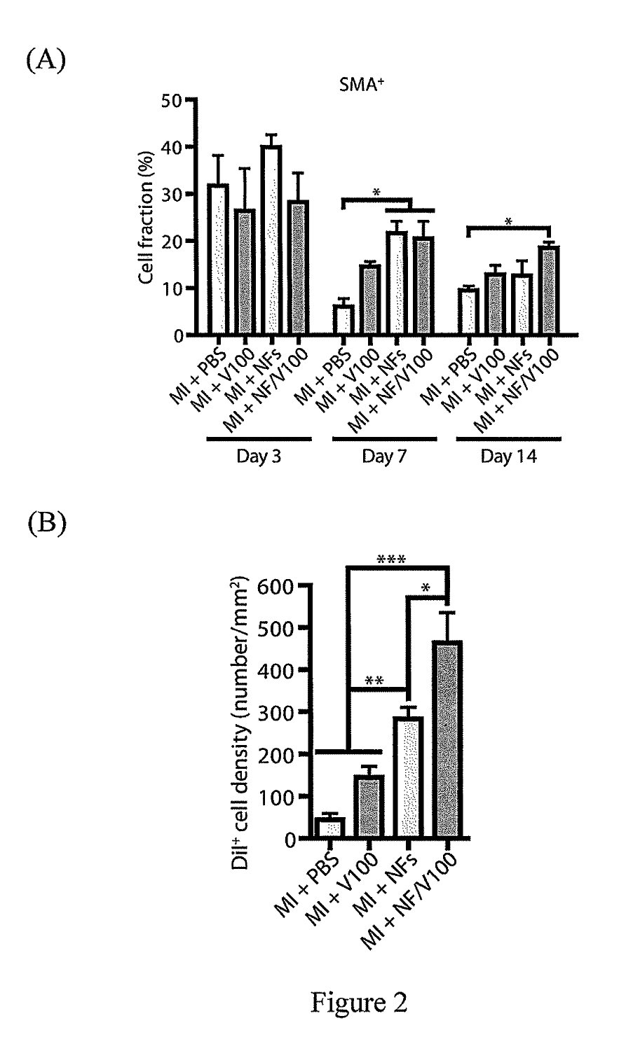

MI was induced in rats, followed with intramyocardial injection of PBS, V100, NFs or NF/V100. The rats were sacrificed on day 3, 7 or 14 post-MI. Then, immunostaining of smooth muscle cells using anti-α smooth muscle actin (α-SMA⁺) antibody (Sigma-Aldrich) was conducted, as shown in FIG. 2A.

α-SMA⁺ myofibroblasts were recruited in the infarcted myocardium of all groups on day 3 post-MI. Myofibroblasts were recruited into the myocardium on day 7 post-injection of NFs, with or without VEGF (MI+NFs and MI+NFs/V100 groups), which implies that myofibroblasts were mostly acquired through the microenvironmental benefits gained by the NFs of the present invention rather than the influence of prolonged VEGF delivery. Interestingly, on day 14 post-MI, the NF/VEGF group also recruited more α-SMA⁺ cells that were capable of vessel integration, and this suggests that the recruited myofibroblasts had differentiated into mural cells, which enveloped nascent capillaries. This process is required for arteriogenesis. Together these results support that for functional arteriogenesis it requires not only VEGF-dependent angiogenesis but also mural cells recruitment by NF injection. Therefore, the NF/VEGF composition of the present invention not only promotes arteriogenesis, but also establishes an intramyocardial microenvironment suitable for myofibroblast engraftment.

A further study was then designed to verify the capacity of the self-assembling peptide of the present invention (i.e., NFs) to capture circulating bone marrow cells (BMCs). BMCs are the main source of myofibroblasts. Rats received intravenous injections of 1×10⁷ allogeneic DiI-labeled BMCs on day 7 post-MI along with intramyocardial injection of PBS, V100, NFs or NF/V100. The rats were sacrificed 1 day after BMC injection, and then the hearts were embedded and sectioned for fluorescence immuno-staining. The results are shown in FIG. 2B.

More DiI⁺ BMCs were retained within the myocardium that received injections of NFs. Notably, the level of myocardial DiI⁺ BMC infiltration was significantly enhanced by NF/VEGF injection. This result suggests that NF/VEGF injection of the present invention creates an intramyocardial microenvironment capable of recruiting circulating BMCs to promote arteriogenesis.

### Example 5

### The NF/VEGF Composition of the Present Invention Recruits Cardiomyocyte-Like Cells

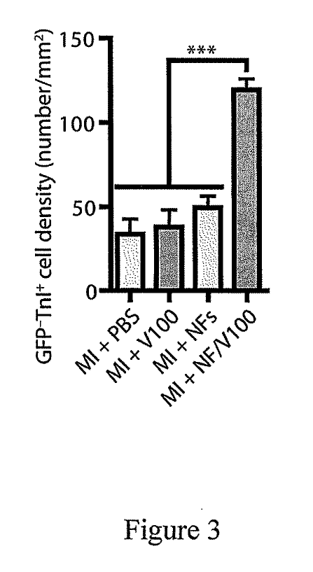

Cardiac troponin-I-positive (cTnI⁺) cells or putative renewed cardiomyocyte-like cells were detected within the injected myocardium on day 28 post-MI following the NF/V100 injection. To test whether these cardiomyocyte-like cells were derived from either non-myogenic stein/progenitor cells or just debris of remnant cardiomyocytes, a genetic fate-mapping approach was conducted using Mer-Cre-Mer/ZEG mice which were hybridized from the cardiac-specific inducible Cre mice (the Mer-Cre-Mer mice) and a dual reporter ZEG transgenic mice. Through induction of Tamoxifen, cardiomyocytes were marked with green fluorescence protein (GFP). On the contrary, stem cell-derived new cardiomyocytes were not marked by GFP. The Mer-Cre-Mer/ZEG mice were injected with 15 μL of PBS, V100, NFs or NF/V100 after experimental MI. On day 28 post-MI, mice were sacrificed and the hearts were sectioned and stained by cTnI antibody. The GFP (green) and cTnI (red) fluorescence signals were observed using fluorescence microscopy (GFP: Abeam; cTnI: DSHB), in which GFP-negative and eTnI-positive cells were stem/progenitor cell origin. The results are shown in FIG. 3.

As shown in FIG. 3, hearts from the NF/VEGF injection group recruited more GFP⁻/cTnI⁺ cardiomyocyte-like cells than the other groups. There were also GFP-negative mature cardiomyocytes observed in the MI+NF/V100 group with no significant difference compared with that in the control. Again, these results suggest that NF/VEGF injection provides an intramyocardial microenvironment favorable for the induction of endogenous cardiomyocyte regeneration.

In summary, the NF/VEGF composition of the present invention can provide a controlled local delivery of VEGF in the heart without vascular leakage in other organs. Thus, it has high safety and effectiveness. In addition, the NF/VEGF composition of the present invention effectively improves cardiac functions and decreases infarct size after MI. These benefits may be through recruitment of myofibroblasts for arteriogenesis and cardiomyocyte-like cells for regeneration. Therefore, it has a great potential for clinical uses.

### REFERENCES

1. Roger V L, et al. (2011) Heart disease and stroke statistics-2011 update: a report from the American heart association. Circulation 123(4):e18-e209.
2. Jessup M & Brozena S (2003) Heart failure. N. Engl. J. Med. 348(20):2007-2018.
3. Laflamme M A, Zbinden S, Epstein S E, & Murry C E (2007) Cell-based therapy for myocardial ischemia and infarction: pathophysiological mechanisms. Annu. Rev. Pathol. Mech. Dis. 2(1):307-339.
4. Ferrara N (2004) Vascular endothelial growth factor: basic science and clinical progress. Endocr. Rev. 25(4):581-611.
5. Coultas L, Chawengsaksophak K, & Rossant J (2005) Endothelial cells and VEGF in vascular development. Nature 438(7070):937-945.
6. Olsson A-K, Dimberg A, Kreuger T, & Claesson-Welsh L (2006) VEGF receptor signalling—in control of vascular function. Nat. Rev. Mol. Cell Biol. 7(5):359-371.
7. Ylä-Herttuala S, Rissanen T T, Vajanto I, & Hartikainen J (2007) Vascular endothelial growth factors: biology and current status of clinical applications in cardiovascular medicine. J. Am. Coll. Cardiol. 49(10):1015-1026.
8. Springer M L, Chen A S, Kraft P E, Bednarski M, & Blau H M (1998) VEGF gene delivery to muscle: potential role for vasculogenesis in adults. Mol. Cell 2(5):549-558.
9. Lee R J, et al. (2000) VEGF gene delivery to myocardium: deleterious effects of unregulated expression. Circulation 102(8):898-901.
10. Ozawa C R, et al. (2004) Microenvironmental VEGF concentration, not total dose, determines a threshold between normal and aberrant angiogenesis. J. Clin. Invest. 113(4):516-527.
11. von Degenfeld G, et al. (2006) Microenvironmental VEGF distribution is critical for stable and functional vessel growth in ischemia. FASEB J. 20(14):2657-2659.
12. Carmeliet P (2003) Angiogenesis in health and disease. Nat. Med. 9(6):653-660.
13. Frontini M J, et al. (2011) Fibroblast growth factor 9 delivery during angiogenesis produces durable, vasoresponsive microvessels wrapped by smooth muscle cells. Nat. Biotechnol. 29(5):421-427.
14. Koike N, et al. (2004) Tissue engineering: creation of long-lasting blood vessels. Nature 428(6979):138-139.
15. Jain R K (2003) Molecular regulation of vessel maturation. Nat. Med. 9(6):685-693.
16. Griffith L G & Swartz M A (2006) Capturing complex 3D tissue physiology in vitro. Nat, Rev. Mol. Cell Biol. 7(3):211-224.
17. Ferreira L, Karp J M, Nobre L, & Langer R (2008) New opportunities: the use of nanotechnologies to manipulate and track stem cells. Cell Stem Cell 3(2):136-146.
18. Discher D E, Mooney D J, & Zandstra P W (2009) Growth factors, matrices, and forces combine and control stem cells. Science 324(5935):1673-1677.
19. Huebsch N & Mooney D J (2009) Inspiration and application in the evolution of biomaterials. Nature 462(7272):426-432.
20. Chien K R, Domian I J, & Parker K K (2008) Cardiogenesis and the complex biology of regenerative cardiovascular medicine. Science 322(5907):1494-1497.
21. Place E S, Evans N D, & Stevens M M (2009) Complexity in biomaterials for tissue engineering. Nat. Mater. 8(6):457-470.
22. Dvir T, Timko B P, Kohane D S, & Langer R (2011) Nanotechnological strategies for engineering complex tissues. Nat. Nanotechnol. 6(1):13-22.

What is claimed is:

1. A pharmaceutical composition for promoting arteriogenesis, comprising:
(1) an effective amount of a drug for promoting arteriogenesis selected from the group consisting of vascular endothelial growth factor (VEGF), fibroblast growth factor (FGF), granulocyte colony-stimulating factor (GCSF), granulocyte-macrophage colony stimulating factor (GM-CSF), hepatocyte growth factor (HGF), angiopoietin, epidermal growth factor (EGF), nerve growth factor (NGF), transforming growth factor (TGF), platelet-derivated growth factor (PDGF), insulin-like growth factor (IGF), brain-derived neurotrophic factor (BDNF), keratinocyte growth factor (KGF), bone morphogenetic protein (BMP), erythropoietin (EPO) and placental growth factor (PIGF); and
(2) a peptide hydrogel comprising a self-assembling peptide selected from the group consisting of the following sequences:
(SEQ ID NO: 1)
AcN-AKAKAEAEAKAKAEAE-NH₂;
(SEQ ID NO: 2)
AcN-AKAEAKAEAKAEAKAE-NH₂;
(SEQ ID NO: 3)
AcN-EAKAEAKAEAKAEAKA-NH₂;
(SEQ ID NO: 4)
AcN-KAEAKAEAKAEAKAEA-NH₂;
(SEQ ID NO: 5)
AcN-AEAKAEAKAEAKAEAK-NH₂;
(SEQ ID NO: 6)
AcN-ADADARARADADARAR-NH₂;
(SEQ ID NO: 7)
AcN-ARADARADARADARAD-NH₂;
(SEQ ID NO: 8)
AcN-DARADARADARADARA-NH₂;
(SEQ ID NO: 9)
AcN-RADARADARADARADA-NH₂;
(SEQ ID NO: 10)
AcN-ADARADARADARADAR-NH₂;
(SEQ ID NO: 11)
AcN-ARADAKAEARADAKAE-NH₂;
(SEQ ID NO: 12)
AcN-AKAEARADAKAEARAD-NH₂;
(SEQ ID NO: 13)
AcN-ARAKADAEARAKADAE-NH₂;
(SEQ ID NO: 14)
AcN-AKARAEADAKARADAE-NH₂;
(SEQ ID NO: 15)
AcN-AQAQAQAQAQAQAQAQ-NH₂;
(SEQ ID NO: 16)
AcN-VQVQVQVQVQVQVQVQ-NH₂;
(SEQ ID NO: 17)
AcN-YQYQYQYQYQYQYQYQ-NH₂;
(SEQ ID NO: 18)
AcN-HQHQHQHQHQHQHQHQ-NH₂;
(SEQ ID NO: 19)
AcN-ANANANANANANANAN-NH₂;
(SEQ ID NO: 20)
AcN-VNVNVNVNVNVNVNVN-NH₂;
(SEQ ID NO: 21)
AcN-YNYNYNYNYNYNYNYN-NH₂;
(SEQ ID NO: 22)
AcN-HNHNHNHNHNHNHNHN-NH₂;
(SEQ ID NO: 23)
AcN-ANAQANAQANAQANAQ-NH₂;
(SEQ ID NO: 24)
AcN-AQANAQANAQANAQAN-NH₂;
(SEQ ID NO: 25)
AcN-VNVQVNVQVNVQVNVQ-NH₂;
(SEQ ID NO: 26)
AcN-VQVNVQVNVQVNVQVN-NH₂;
(SEQ ID NO: 27)
AcN-YNYQYNYQYNYQYNYQ-NH₂;
(SEQ ID NO: 28)
AcN-YQYNYQYNYQYNYQYN-NH₂;
(SEQ ID NO: 29)
AcN-HNHQHNHQHNHQHNHQ-NH₂;
(SEQ ID NO: 30)
AcN-HQHNHQHNHQHNHQHN-NH₂;
(SEQ ID NO: 31)
AcN-AKAQADAKAQADAKAQAD-NH₂;
(SEQ ID NO: 32)
AcN-VKVQVDVKVQVDVKVQVD-NH₂;
(SEQ ID NO: 33)
AcN-YKYQYDYKYQYDYKYQYD-NH₂;
(SEQ ID NO: 34)
AcN-HKHQHDHKHQHDHKHQHD-NH₂;
(SEQ ID NO: 35)
AcN-RARADADARARADADA-NH₂;
(SEQ ID NO: 36)
AcN-RADARGDARADARGDA-NH₂;
(SEQ ID NO: 37)
AcN-RAEARAEARAEARAEA-NH₂;
(SEQ ID NO: 38)
AcN-KADAKADAKADAKADA-NH₂;
(SEQ ID NO: 39)
AcN-AEAEAHAHAEAEAHAH-NH₂;
(SEQ ID NO: 40)
AcN-FEFEFKFKFEFEFKFK-NH₂;
(SEQ ID NO: 41)
AcN-LELELKLKLELELKLK-NH₂;
(SEQ ID NO: 42)
AcN-AEAEAKAKAEAEAKAK-NH₂;
(SEQ ID NO: 43)
AcN-AEAEAEAEAKAK-NH₂;
(SEQ ID NO: 44)
AcN-KAKAKAKAEAEAEAEA-NH₂;
(SEQ ID NO: 45)
AcN-AEAEAEAEAKAKAKAK-NH₂;
(SEQ ID NO: 46)
AcN-RARARARADADADADA-NH₂;
(SEQ ID NO: 47)
AcN-ADADADADARARARAR-NH₂;
(SEQ ID NO: 48)
AcN-DADADADARARARARA-NH₂;
(SEQ ID NO: 49)
AcN-HEHEHKHKHEHEHKHK-NH₂;
(SEQ ID NO: 50)
AcN-VEVEVEVEVEVEVEVEVEVE-NH₂;
and
(SEQ ID NO: 51)
AcN-RFRFRFRFRFRFRFRFRFRF-NH₂;
in which the right end of said sequences is not —CNH₂, and the pharmaceutical composition forms a microenvironment for autologous cell recruitment and tissue regeneration.

2. The pharmaceutical composition according to claim 1, wherein said self-assembling peptide is AcN-RARADADARARADADA-NH₂ (SEQ ID NO: 35).

3. The pharmaceutical composition according to claim 1, wherein said peptide hydrogel is used as a carrier for loading said drug.

4. The pharmaceutical composition according to claim 1, wherein said peptide hydrogel is composed of said self-assembling peptide and a buffer solution.

5. The pharmaceutical composition according to claim 3, wherein said peptide hydrogel comprises 0.1% to 10% by weight of said self-assembling peptide.

6. The pharmaceutical composition according to claim 3, wherein said buffer solution is water, saline, or a buffer solution comprising elements needed for peptide self-assembly, specifically phosphate buffer solution.

7. The pharmaceutical composition according to claim 1, wherein said drug is physically connected with said peptide hydrogel.

8. The pharmaceutical composition according to claim 6, wherein said drug is vascular endothelial growth factor (VEGF).

9. The pharmaceutical composition according to claim 1, wherein said tissue regeneration is tissue regeneration of heart, liver, spleen, lung, kidney, brain, pancreas, eye, cartilage, urinary bladder or muscle.

10. The pharmaceutical composition according to claim 1, which is used to treat a cardiovascular disease.

11. The pharmaceutical composition according to claim 9, wherein said cardiovascular disease comprises myocardial infarction, heart failure, ischemic heart diseases, stroke and peripheral vascular diseases.

12. The pharmaceutical composition according to claim 1, which is administered by injection into myocardium through thoracotomy, cardiac catheterization, echo-guided injection, or any other methods for injection.

13. The pharmaceutical composition according to claim 1, wherein said autologous cell is myofibroblast, bone marrow cell, cardiomyocyte-like cell, precursor cell and/or stem cell.

14. A method for preparing the pharmaceutical composition according to claim 1, comprising the following steps:
(a) preparing a peptide hydrogel from a self-assembling peptide and a buffer solution, wherein said self-assembling peptide is the self-assembling peptide as defined in claim 1; and
(b) mixing an effective amount of a drug for promoting arteriogenesis and said peptide hydrogel to obtain the pharmaceutical composition according to claim 1, in which said drug for promoting arteriogenesis is the drug for promoting arteriogenesis as defined in claim 1.

15. The method according to claim 14, wherein said self-assembling peptide is AcN-RARADADARARADADA-NH₂ (SEQ ID NO: 35).

16. The method according to claim 14, wherein said peptide hydrogel comprises 0.1% to 10% by weight of said self-assembling peptide.

17. The method according to claim 14, wherein said buffer solution is water, saline, or a buffer solution comprising elements needed for peptide self-assembly.

18. The method according to claim 14, wherein said peptide hydrogel is prepared by mixing through sonication or any other process for mixing the peptide hydrogel in the step (1).

19. The method according to claim 14, wherein said drug is vascular endothelial growth factor (VEGF).

20. A method for treating a cardiovascular disease, comprising applying the pharmaceutical composition according to claim 1 to a subject in need.

21. The method according to claim 20, wherein said cardiovascular disease comprises myocardial infarction, heart failure, ischemic heart diseases, stroke and peripheral vascular diseases.


SEQUENCE LISTING (51 sequences)

SEQ ID NO: 1 - protein, 16 aa, Artificial
  synthetic sequence
AKAKAEAEAKAKAEAE

SEQ ID NO: 2 - protein, 16 aa, Artificial
  synthetic sequence
AKAEAKAEAKAEAKAE

SEQ ID NO: 3 - protein, 16 aa, Artificial
  synthetic sequence
EAKAEAKAEAKAEAKA

SEQ ID NO: 4 - protein, 16 aa, Artificial
  synthetic sequence
KAEAKAEAKAEAKAEA

SEQ ID NO: 5 - protein, 16 aa, Artificial
  synthetic sequence
AEAKAEAKAEAKAEAK

SEQ ID NO: 6 - protein, 16 aa, Artificial
  synthetic sequence
ADADARARADADARAR

SEQ ID NO: 7 - protein, 16 aa, Artificial
  synthetic sequence
ARADARADARADARAD

SEQ ID NO: 8 - DNA, 16 bp, Artificial
  synthetic sequence
daradaradaradara

SEQ ID NO: 9 - protein, 16 aa, Artificial
  synthetic sequence
RADARADARADARADA

SEQ ID NO: 10 - protein, 16 aa, Artificial
  synthetic sequence
ADARADARADARADAR

SEQ ID NO: 11 - protein, 16 aa, Artificial
  synthetic sequence
ARADAKAEARADAKAE

SEQ ID NO: 12 - protein, 16 aa, Artificial
  synthetic sequence
AKAEARADAKAEARAD

SEQ ID NO: 13 - protein, 16 aa, Artificial
  synthetic sequence
ARAKADAEARAKADAE

SEQ ID NO: 14 - protein, 16 aa, Artificial
  synthetic sequence
AKARAEADAKARADAE

SEQ ID NO: 15 - protein, 16 aa, Artificial
  synthetic sequence
AQAQAQAQAQAQAQAQ

SEQ ID NO: 16 - protein, 16 aa, Artificial
  synthetic sequence
VQVQVQVQVQVQVQVQ

SEQ ID NO: 17 - protein, 16 aa, Artificial
  synthetic sequence
YQYQYQYQYQYQYQYQ

SEQ ID NO: 18 - protein, 16 aa, Artificial
  synthetic sequence
HQHQHQHQHQHQHQHQ

SEQ ID NO: 19 - protein, 16 aa, Artificial
  synthetic sequence
ANANANANANANANAN

SEQ ID NO: 20 - protein, 16 aa, Artificial
  synthetic sequence
VNVNVNVNVNVNVNVN

SEQ ID NO: 21 - protein, 16 aa, Artificial
  synthetic sequence
YNYNYNYNYNYNYNYN

SEQ ID NO: 22 - protein, 16 aa, Artificial
  synthetic sequence
HNHNHNHNHNHNHNHN

SEQ ID NO: 23 - protein, 16 aa, Artificial
  synthetic sequence
ANAQANAQANAQANAQ

SEQ ID NO: 24 - protein, 16 aa, Artificial
  synthetic sequence
AQANAQANAQANAQAN

SEQ ID NO: 25 - protein, 16 aa, Artificial
  synthetic sequence
VNVQVNVQVNVQVNVQ

SEQ ID NO: 26 - protein, 16 aa, Artificial
  synthetic sequence
VQVNVQVNVQVNVQVN

SEQ ID NO: 27 - protein, 16 aa, Artificial
  synthetic sequence
YNYQYNYQYNYQYNYQ

SEQ ID NO: 28 - protein, 16 aa, Artificial
  synthetic sequence
YQYNYQYNYQYNYQYN

SEQ ID NO: 29 - protein, 16 aa, Artificial
  synthetic sequence
HNHQHNHQHNHQHNHQ

SEQ ID NO: 30 - protein, 16 aa, Artificial
  synthetic sequence
HQHNHQHNHQHNHQHN

SEQ ID NO: 31 - protein, 18 aa, Artificial
  synthetic sequence
AKAQADAKAQADAKAQAD

SEQ ID NO: 32 - protein, 18 aa, Artificial
  synthetic sequence
VKVQVDVKVQVDVKVQVD

SEQ ID NO: 33 - protein, 18 aa, Artificial
  synthetic sequence
YKYQYDYKYQYDYKYQYD

SEQ ID NO: 34 - protein, 18 aa, Artificial
  synthetic sequence
HKHQHDHKHQHDHKHQHD

SEQ ID NO: 35 - protein, 16 aa, Artificial
  synthetic sequence
RARADADARARADADA

SEQ ID NO: 36 - protein, 16 aa, Artificial
  synthetic sequence
RADARGDARADARGDA

SEQ ID NO: 37 - protein, 16 aa, Artificial
  synthetic sequence
RAEARAEARAEARAEA

SEQ ID NO: 38 - protein, 16 aa, Artificial
  synthetic sequence
KADAKADAKADAKADA

SEQ ID NO: 39 - protein, 16 aa, Artificial
  synthetic sequence
AEAEAHAHAEAEAHAH

SEQ ID NO: 40 - protein, 16 aa, Artificial
  synthetic sequence
FEFEFKFKFEFEFKFK

SEQ ID NO: 41 - protein, 16 aa, Artificial
  synthetic sequence
LELELKLKLELELKLK

SEQ ID NO: 42 - protein, 16 aa, Artificial
  synthetic sequence
AEAEAKAKAEAEAKAK

SEQ ID NO: 43 - protein, 12 aa, Artificial
  synthetic sequence
AEAEAEAEAKAK

SEQ ID NO: 44 - protein, 16 aa, Artificial
  synthetic sequence
KAKAKAKAEAEAEAEA

SEQ ID NO: 45 - protein, 16 aa, Artificial
  synthetic sequence
AEAEAEAEAKAKAKAK

SEQ ID NO: 46 - protein, 16 aa, Artificial
  synthetic sequence
RARARARADADADADA

SEQ ID NO: 47 - protein, 16 aa, Artificial
  synthetic sequence
ADADADADARARARAR

SEQ ID NO: 48 - protein, 16 aa, Artificial
  synthetic sequence
DADADADARARARARA

SEQ ID NO: 49 - protein, 16 aa, Artificial
  synthetic sequence
HEHEHKHKHEHEHKHK

SEQ ID NO: 50 - protein, 20 aa, Artificial
  synthetic sequence
VEVEVEVEVEVEVEVEVEVE

SEQ ID NO: 51 - protein, 20 aa, Artificial
  synthetic sequence
RFRFRFRFRFRFRFRFRFRF